(19) United States
(12) Patent Application Publication
(10) Pub. No.: US 2019/0002471 A1
(43) Pub. Date: Jan. 3, 2019

(54) Crystalline Form Of Ticagrelor

(71) Applicant: Amneal Pharmaceuticals Company GmbH, Steinhausen (CH)

(72) Inventors: Virendra Kumar Agarwal, Ahmedabad, Gujarat (IN); Lalit Keshav Katariya, Ahmedabad, Gujarat (IN); Kamalakar Gangireddy Reddy, Ahmedabad, Gujarat (IN); Gaurav Bhogilal Patel, Ahmedabad, Gujarat (IN); Chandrakant Bhagvanbhai Patel, Ahmedabad, Gujarat (IN); Ankur Amrutbhai Kaneria, Gujarat (IN); Hitesh Sureshbhai Patel, Ahmedabad, Gujarat (IN)

(21) Appl. No.: 16/066,425

(22) PCT Filed: Jan. 3, 2017
(86) PCT No.: PCT/EP2017/050087

§ 371 Date: Jun. 27, 2018

(30) Foreign Application Priority Data
Jan. 5, 2016 (IN) 201621000333

(51) Int. Cl. C07D 487/04

(52) U.S. Cl. CPC C07D 487/04; C07B 2200/13

(57) ABSTRACT

A crystalline form of ticagrelor, designated as Form-AM, has a defined interplanar spacing as illustrated by FIG. 1.

### FIELD OF INVENTION

The present invention relates to a crystalline form of ticagrelor, designated as Form-AM.

### BACKGROUND OF INVENTION

Ticagrelor is covered under class of drug cyclopentyltriazolopyrimidine which is inhibitor of platelet activation and aggregation mediated by the P2Y₁₂ ADP-receptor. Ticagrelor is indicated to reduce the rate of thrombotic cardiovascular events in patients with acute coronary syndrome (ACS). Ticagrelor is known by chemical name (1S,2S,3R,5S)-3-[7-{[(1R,2S)-2-(3,4-difluorophenyl)cyclopropyl]amino}-5-(propylthio)-3H-[1,2,3]-triazolo[4,5-d]pyrimidin-3-yl]-5-(2-hydroxyethoxy)cyclopentane-1,2-diol. Ticagrelor is marketed in USA by Astrazeneca under trade name Brilinta® in the form of oral tablet of 90 mg dosage form. Ticagrelor is represented by following structure.

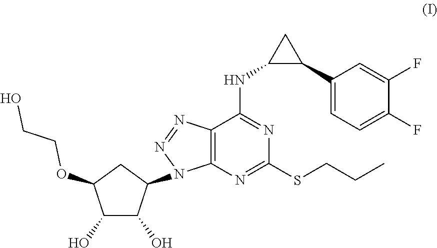 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

Ticagrelor is disclosed first in U.S. Pat. No. 6,525,060 B1. This patent discloses process for preparation of ticagrelor. However, this patent is silent about polymorphic form.

U.S. Pat. No. 7,265,124 B2 discloses polymorphic Form I, II, III and IV and mixture of forms of ticagrelor which are anhydrous form. It also discloses α-form and amorphous form. It discloses various hydrated forms of ticagrelor varying in water content including hemihydrate.

Polymorphism is an important aspect of pharmaceutical drug in terms of its solubility and bioavailability. One of the most important physical properties of pharmaceutical compounds is their solubility in aqueous solution, particularly their solubility in the gastric juices of a patient. Different crystalline forms of polymorphs of the same pharmaceutical compounds can and reportedly do have different aqueous solubility. The different solubility of the drug compound affects the bioavailability of drug at target site.

In the formulation of drug compositions, it is important for the drug substance to be in a form in which it can be conveniently handled and processed. This is of importance, not only from the point of view of obtaining a commercially viable manufacturing process, but also from the point of subsequent manufacture of pharmaceutical formulation comprising the active compound. Chemical stability, solid state stability and shelf life of the active ingredients are also very important factors. The drug substance and compositions containing it should be capable of being effectively stored over appreciable periods of time without exhibiting a significant change in the active component's physico-chemical characteristics (e.g. its chemical composition, density, hygroscopicity and solubility). Moreover, it is also important to be able to provide drug in a form which is as pure as possible. Amorphous materials may present problems such as unreliable solubility and lower stability which may lead to impurity. The skilled person will appreciate that, if a drug can be readily obtained in a stable crystalline form, the above problems may be solved. Thus, in the manufacture of commercially viable and pharmaceutically acceptable, drug compositions, it is desirable, wherever possible, to provide drug in a substantially crystalline, and stable, form.

The present invention provides novel crystalline Form-AM of ticagrelor and a process for its preparation.

### SUMMARY OF THE INVENTION

In an aspect, the present invention provides a crystalline form of ticagrelor, designated as Form AM.

In one aspect, the present provides a process for the preparation of a novel crystalline Form-AM of ticagrelor (I).

In one aspect, the present invention provides a process for the preparation of a novel crystalline Form-AM of ticagrelor (I):

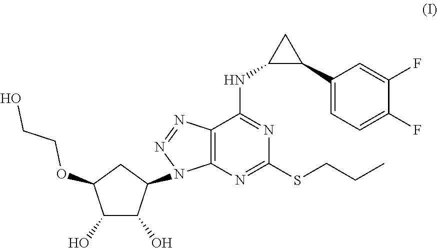 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

which comprises,
a) preparing a solution of ticagrelor in aqueous ester solvent or taking ticagrelor solution in aq. ester solvent obtained from reaction mixture;
b) adding the above solution to aliphatic hydrocarbon or ether, containing seed of ticagrelor Form-AM; and
c) isolating crystalline Form-AM of ticagrelor.

In another aspect, the present invention provides a process for the preparation of a novel crystalline Form-AM of ticagrelor (I):

which comprises,
p) preparing a solution of ticagrelor in aqueous ethyl acetate or taking ticagrelor solution in aqueous ethyl acetate obtained from reaction mixture;
q) adding the above solution to cyclohexane, containing seed of ticagrelor Form-AM; and
r) isolating crystalline Form-AM of ticagrelor.

In yet another aspect, the present invention provides a process for preparation of a novel crystalline Form-AM of ticagrelor (I):

which comprises,
i) reducing [(4-{7-[2-(3,4-Difluoro-phenyl)-cyclopropylamino]-5-propylsulfanyl-[1,2,3] triazolo[4,5-d]pyrimidin-3-yl}-2,3-dihydroxy-cyclopentyloxy)-acetic acid ethyl ester of formula II;
ii) adding acid, solvent and water to reaction mixture, extracting and separating organic phase;
iii) water, methanol and ethyl acetate is added to the reaction mixture, extracting and separating organic phase;
iv) removing solvent from organic phase to give residue;
v) purification of residue using ethyl acetate and cyclohexane; and
vi) re-purification of residue using aq. ethyl acetate, cyclohexane and seed of a novel crystalline Form-AM of ticagrelor (I).

### BRIEF DESCRIPTION OF FIGURE

FIG. 1: XRPD of crystalline Form-AM of ticagrelor as obtained in example 1.

### DETAIL DESCRIPTION OF INVENTION

The term “about” when used in the present invention preceding a number and referring to it, is meant to designate any value which lies within the range of ±10%, preferably within a range of ±5%, more preferably within a range of ±2%, still more preferably within a range of ±1% of its value. For example “about 10” should be construed as meaning within the range of 9 to 11, preferably within the range of 9.5 to 10.5, more preferably within the range of 9.8 to 10.2, and still more preferably within the range of 9.9 to 10.1.

For XRD, the relative intensities of the peaks can vary, depending upon the sample preparation technique, the sample mounting procedure and the particular instrument employed.

In an aspect, the present invention provides a crystalline form of ticagrelor, designated as Form-AM having a typical x-ray powder diffraction pattern as represented by the following interplanar spacing:

| d-Spacing (Å) | Relative Intensity (%) |
|---|---|
| 16.4340 | 100.00 |
| 16.0673 | 70.19 |
| 15.1372 | 12.56 |
| 13.2449 | 23.90 |
| 12.6933 | 3.19 |
| 8.4139 | 9.54 |
| 8.0310 | 6.72 |
| 7.9460 | 5.67 |
| 7.6708 | 3.58 |
| 7.1140 | 15.66 |
| 6.6158 | 61.76 |
| 6.3688 | 34.71 |
| 6.0017 | 8.99 |
| 5.3655 | 1.96 |
| 5.4687 | 3.54 |
| 5.3564 | 6.84 |
| 5.2487 | 12.60 |
| 5.1053 | 30.09 |
| 4.9713 | 14.35 |
| 4.8773 | 71.97 |
| 4.7100 | 16.81 |
| 4.6528 | 20.10 |
| 4.5686 | 7.39 |
| 4.5178 | 6.93 |
| 4.4123 | 8.68 |
| 4.1952 | 22.30 |
| 4.1799 | 25.85 |
| 4.1353 | 11.92 |
| 4.0364 | 15.73 |
| 3.9442 | 43.85 |
| 3.8398 | 6.56 |
| 3.6868 | 63.55 |
| 3.5004 | 6.65 |
| 3.4444 | 6.38 |
| 3.1346 | 11.12 |
| 3.2454 | 3.07 |
| 3.1651 | 7.36 |
| 3.0957 | 3.23 |
| 2.9938 | 5.70 |
| 2.8828 | 6.75 |
| 2.7485 | 2.63 |
| 2.6718 | 2.91 |
| 2.6424 | 3.09 |
| 2.6055 | 2.17 |
| 2.3481 | 2.41 |
| 2.3027 | 2.36 |
| 2.2561 | 1.55 |
| 2.2042 | 3.39 |
| 2.0677 | 1.66 |

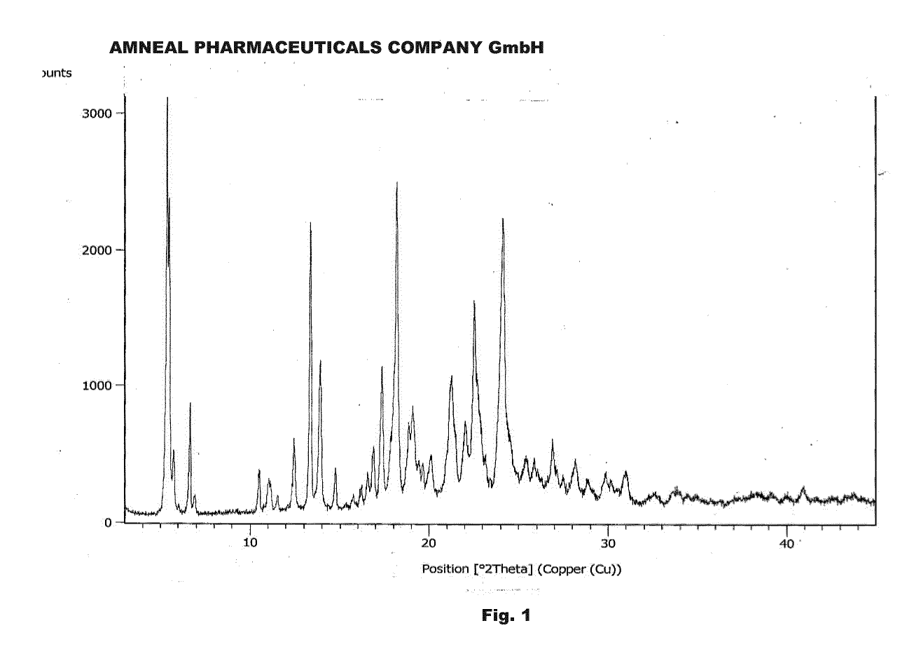

FIG. 1 shows typical X-ray powder diffraction pattern of “Form AM”. In an aspect, the present invention provides a crystalline form of ticagrelor, designated as Form AM, characterized by an X-ray powder diffraction pattern substantially as illustrated by FIG. 1.

In one aspect, the present invention provides a process for the preparation of a novel crystalline Form-AM of ticagrelor (I):

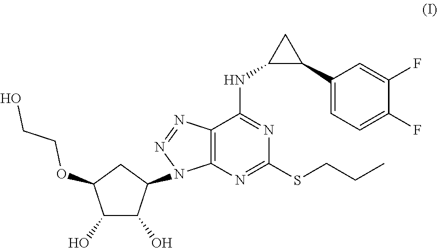 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

which comprises,
a) preparing a solution of ticagrelor in aqueous ester solvent or taking ticagrelor solution in aqueous ester solvent obtained from reaction mixture;
b) adding the above solution to aliphatic hydrocarbon or ether, containing seed of ticagrelor Form-AM; and
c) isolating crystalline Form-AM of ticagrelor.

Crystalline Form-AM of ticagrelor can be prepared by dissolving ticagrelor in aqueous ester solvent. The aqueous solution of ester solvent varies from 1% to 25% water in ester solvent, preferably 5% water in ester solvent. In this process, solvent containing product is added to anti-solvent. This process is reverse addition method compared to conventional method of solvent/anti-solvent wherein anti-solvent is added to solvent containing product. In the reverse addition method, seed crystal of desired form of compound is taken in anti-solvent to which solvent containing compound is added which upon crystallization gives desired form of compound.

In the process of present invention, the ester solvent is selected from solvent such as ethyl acetate, methyl acetate, isopropyl acetate and the like. Anti-solvent is selected from hydrocarbon solvent such as cyclohexane, hexane, heptane and the like; ether such as petroleum ether, diethyl ether, methyl tert-butyl ether, diisopropyl ether and the like. In this process, anti-solvent is taken in reaction vessel. Seed quantity of ticagrelor Form-AM is added into anti-solvent. The seed quantity is taken in ratio of 0.5 to 10% compared to input quantity of ticagrelor. The ratio of solvent to anti-solvent varies in the range of 1:1 to 1:10, preferably 1:5.

Preferably, the present invention provides a process for the preparation of a novel crystalline Form-AM of ticagrelor (I), which comprises:
p) preparing a solution of ticagrelor in aqueous ethyl acetate or taking ticagrelor solution in aqueous ethyl acetate obtained from reaction mixture;
q) adding the above solution to cyclohexane containing seed of ticagrelor Form-AM; and
r) isolating crystalline Form-AM of ticagrelor.

Crystalline Form-AM of ticagrelor can be prepared by dissolving ticagrelor in aqueous ethyl acetate. The aq. solution of ethyl acetate varies from 1% to 25% water in ethyl acetate, preferably 5% water in ethyl acetate.

In another embodiment, the present invention provides a process for preparation of a novel crystalline Form-AM of ticagrelor (I):

comprises,
i) reducing [(4-{7-[2-(3,4-Difluoro-phenyl)-cyclopropylamino]-5-propylsulfanyl-[1,2,3]triazolo[4,5-d]pyrimidin-3-yl}-2,3-dihydroxy-cyclopentyloxy)-acetic acid ethyl ester of formula II;
ii) adding acid, ethyl acetate and water to reaction mixture, extracting and separating organic phase;
iii) adding methanol to the reaction mixture of step (ii), extracting and separating organic phase;
iv) removing solvent from organic phase to give residue;
v) purification of residue using ethyl acetate and cyclohexane; and
vi) re-purification of residue using aqueous ethyl acetate, cyclohexane and seed of a novel crystalline Form-AM of ticagrelor (I) to obtain crystalline Form-AM of ticagrelor (I).

The solid state forms of the present invention may be dried. Drying may be carried out, for example, at elevated temperature with or without reduced pressure.

Drying may be suitably carried out in a tray dryer, vacuum oven, Buchi® Rotavapor®, air oven, fluidized bed dryer, spin flash dryer, flash dryer, cone dryer, agitated nutsche filter cum dryer, nauta dryer or the like or any other suitable dryer.

The drying may be carried out at temperature of less than about 150° C., or less than about 120° C., or less than about 100° C., or less than about 70° C., or less than about 60° C., or less than about 50° C., or less than about 40° C., or less than about 20° C., or less than about 0° C., or less than about −20° C. or any other suitable temperature. The drying may be carried out under reduced pressure, that is, less than standard atmospheric pressure or at atmospheric pressure or any other suitable pressure. The drying may take place over a period of about 30 minutes to about 12 hours, or about 2 hours to about 4 hours, or any other suitable time period.

The dried product may be optionally subjected to techniques such as sieving to get rid of lumps before or after drying. The dried product may be optionally milled to get desired particle sizes. Milling or micronization may be performed before drying, or after the completion of drying of the product. Techniques that may be used for particle size reduction include, without limitation, ball, roller and hammer mills, and jet mills.

In an aspect, ticagrelor may have a D₉₀ particle size of less than about 200 μm, or less than about 150 μm, or less than about 100 μm, or less than about 90 μm, or less than about 80 μm, or less than about 60 μm, or less than about 50 μm, or less than about 40 μm, or less than about 30 μm, or less than about 20 μm, or less than about 10 μm, or less than about 5 μm, or any other suitable particle sizes.

In this embodiment, ticagrelor (I) is obtained as crystalline Form-AM as part of work up and purification procedure done on reduction reaction step in penultimate step. The reaction scheme is as depicted below.

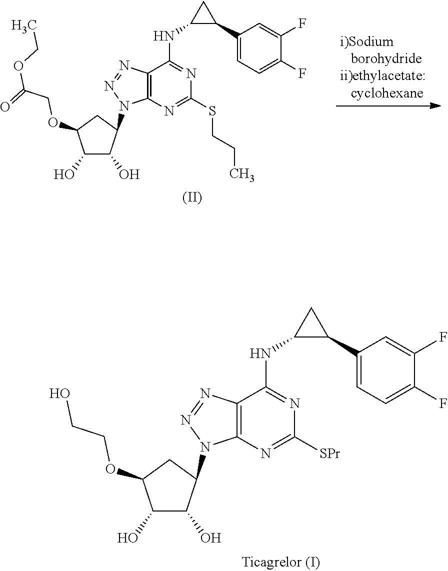

In this process, [(4-{7-[2-(3,4-Difluoro-phenyl)-cyclopropylamino]-5-propylsulfanyl-[1,2,3]triazolo[4,5-d]pyrimidin-3-yl}-2,3-dihydroxy-cyclopentyloxy)-acetic acid ethyl ester of formula II is reduced to get ticagrelor. The reduction reaction is performed in the presence of solvent and reducing agent. The reducing agent is selected from borohydride such as sodium borohydride, sodium cyano borohydride, lithium borohydride, lithium aluminium hydride and the like. The solvent selected remains inert to the reduction condition. The solvent is selected from diglyme, tetrahydrofuran (THE), acetonitrile, alcohol such as methanol, ethanol, propanol, isopropanol and the like or mixture thereof. Acid used is selected from organic acid such as acetic acid, formic acid and the like.

Powder X-ray Diffraction was performed using PANALYTICAL ExpertPro DY666, the powder X-ray diffraction pattern was measured at room temperature using a Cu Kα filled tube (45 kV, 40 mA) as the X-ray source with a wide-angle goniometer, a ½° scattering slit, an programmable divergence slit, and a x'celerator detector. Data collection was done in 2θ continuous scan mode at a scan speed of 0.047747/s in scan steps of 0.0083556 in the range of 3° to 45°. Cu radiation of λ=1.5405 A° was used.

The following examples are given for the purpose of illustrating the present invention and should not be considered as limitation on the scope or spirit of the invention.

### Example 1

### Preparation of Ticagrelor

To a stirred cooled 15-25° C. solution of [(4-{7-[2-(3,4-Difluoro-phenyl)-cyclopropylamino]-5-propylsulfanyl-[1,2,3]triazolo[4,5-d]pyrimidin-3-yl}-2,3-dihydroxy-cyclopentyloxy)-acetic acid ethyl ester (1.0 Kg) in diglyme (10.0 L) was added sodium borohydride (0.134 Kg) in five lots.

The reaction mixture was heated to 50-60° C. and stirred for 3 h. The progress of the reaction was monitored by HPLC. After completion of the reaction, acetic acid (0.5 L) was added dropwise to the reaction mixture and stirred for 15 m. The reaction mixture was added to mixture of water and ethyl acetate (1:2) (30.0 L) and extracted. The organic layer is separated. To the organic layer was added methanol (2.0 L) and aq. potassium bicarbonate solution (10% w/v) (5.0 L) and extracted. The organic layer was separated. The above extraction and separation of organic layer step was repeated twice. The organic layer was washed with brine (10%) (5.0 L) and evaporated to dryness to give residue. The residue was stripped with ethyl acetate (1.0 L). To the residue was added ethyl acetate (3.5 L) and heated to 60-70° C. till dissolve. To the above solution, cyclohexane (5.25 L) was added at the same temperature and stirred for 30 m. The reaction mixture was cooled to 25-35° C. for 3 h. The solid obtained was filtered, washed with mixture of ethyl acetate and cyclohexane (2:3) (1.0 L) and suck dried. The wet cake was stripped with ethyl acetate (2.0 L) to give residue. The residue was dissolved in the mixture of ethyl acetate and water (95:5) (2.2 L). In another round bottom flask cyclohexane (10.5 L) and seed quantity of ticagrelor (Form AM) (0.02 Kg) was taken. Above prepared ethyl acetate solution was added slowly to this mixture at 25-35° C. The reaction mixture was stirred at the same temperature for 4 h. The obtained crystalline solid was filtered, washed with mixture of ethyl acetate and cyclohexane (15:85) (1.0 L), slurry wash with water (10.0 L) and suck dried. The solid was dried in vacuum dryer at 55-65° C. for 12-16 h to give ticagrelor (0.5 Kg) as crystalline Form-AM.

Purity by HPLC: 99.5%

The XRPD of above obtained Ticagrelor is given in FIG. 1.

### Example 2

### Preparation of Ticagrelor Crystalline Form-AM.

Ticagrelor (0.1 Kg) was dissolved in the mixture of ethyl acetate and water (95:5) (0.3 L). In another RBF cyclohexane (1.5 L) and seed quantity of Ticagrelor (Form-AM) (0.002 Kg) was taken. Above prepared ethyl acetate solution was added slowly to this mixture at 25-35° C. The reaction mixture was stirred at the same temperature for 4 h. The obtained crystalline solid was filtered, washed with mixture of ethyl acetate and cyclohexane (15:85) (0.1 L), slurry wash with water (1.0 L) and suck dried. The solid was dried in vacuum dryer at 55-65° C. for 12-16 h to give ticagrelor (0.05 Kg) as crystalline Form-AM.

1. A crystalline Form-AM of ticagrelor (I), having a typical x-ray powder diffraction pattern with d-spacing (A°) as represented by the following interplanar spacing:
d-spacing (A°)
16.4340
16.0673
15.1372
13.2449
12.6933
8.4139
8.0310
7.9460
7.6708
7.1140
6.6158
6.3688
6.0017
5.3655
5.4687
5.3564
5.2487
5.1053
4.9703
4.8773
4.7100
4.6528
4.5686
4.5178
4.4123
4.1952
4.1799
4.1353
4.0364
3.9442
3.8398
3.6868
3.5004
3.4444
3.1346
3.2454
3.1651
3.0957
2.9938
2.8828
2.7485
2.6718
2.6424
2.6055
2.3481
2.3027
2.2561
2.2042
2.0677

2. The crystalline Form-AM of claim 1, characterized by an X-ray powder diffraction pattern substantially as illustrated by FIG. 1.

3. A process for preparation of crystalline Form-AM of ticagrelor (I), which comprises: 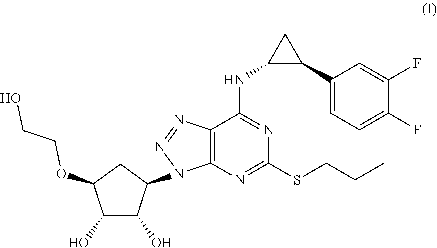 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]
a) preparing a solution of ticagrelor in aqueous ester solvent or taking ticagrelor solution in aqueous ester solvent obtained from reaction mixture;
b) adding the above solution to aliphatic hydrocarbon or ether containing seed of ticagrelor Form-AM; and
c) isolating crystalline Form-AM of ticagrelor.

4. The process of claim 3, wherein the ester solvent is selected from group consisting of ethyl acetate, isopropyl acetate, n-propyl acetate and methyl acetate.

5. The process of claim 3, wherein the aliphatic hydrocarbon is selected from the group consisting of cyclohexane, hexane, and heptane.

6. The process of claim 3, wherein ether is selected from the group consisting of petroleum ether, diethyl ether, methyl tert-butyl ether, and disiopropyl ether.

7. The process of claim 3, which comprises:
p) preparing a solution of ticagrelor in aqueous ethyl acetate or taking ticagrelor solution in aqueous ethyl acetate obtained from reaction mixture;
q) adding the above solution to cyclohexane containing seed of ticagrelor Form-AM; and
r) isolating crystalline Form-AM of ticagrelor.

8. A process for preparation of crystalline Form-AM of ticagrelor (I), comprises: 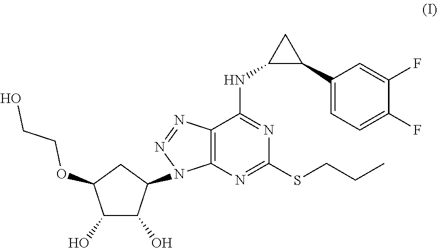 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]
i) reducing [(4-{7-[2-(3,4-Difluoro-phenyl)-cyclopropylamino]-5-propylsulfanyl-[1,2,3]triazolo[4,5-d]pyrimidin-3-yl}-2,3-dihydroxy-cyclopentyloxy)-acetic acid ethyl ester of formula II; 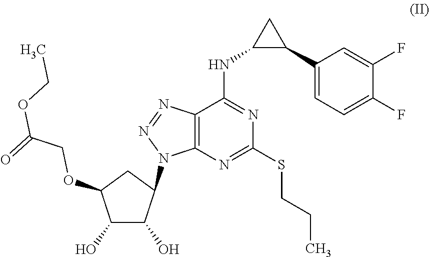 [SMILES: CCCSc1nc(N[C@@H]2C[C@H]2c2ccc(F)c(F)c2)c2nnn([C@@H]3C[C@H](OCC(=O)OCC)[C@@H](O)[C@H]3O)c2n1]
ii) adding acid, ethyl acetate and water to reaction mixture, extracting and separating organic phase;
iii) adding methanol to the reaction mixture of step (ii), extracting and separating organic phase;
iv) removing solvent from organic phase to give residue;
v) purification of residue using ethyl acetate and cyclohexane; and
vi) re-purification of residue using aqueous ethyl acetate, cyclohexane and seed of a novel crystalline Form-AM of ticagrelor (I) to obtain crystalline Form-AM of ticagrelor (I).

9. The process of claim 8, wherein the reducing agents are selected from group consisting of borohydride such as sodium borohydride, sodium cyano borohydride, lithium borohydride and lithium aluminium hydride.

10. The process of claim 8, wherein the acid used is selected from organic acid such as acetic acid and formic acid.

11. The crystalline Form-AM of claim 1, having particle size D₉₀ less than about 200 μm.
